(19) United States
(12) Patent Application Publication
(10) Pub. No.: US 2024/0050038 A1
(43) Pub. Date: Feb. 15, 2024

(54) SELF-POWERED TRIBOELECTRIC MXENE-BASED 3D-PRINTED WEARABLE PHYSIOLOGICAL BIOSIGNAL SENSING SYSTEM FOR ON-DEMAND, WIRELESS, AND REAL-TIME HEALTH MONITORING

(71) Applicant: THE REGENTS OF THE UNIVERSITY OF CALIFORNIA, Oakland, CA (US)

(72) Inventor: Rahim Esfandyarpour, Irvine, CA (US)

(21) Appl. No.: 18/447,884

(22) Filed: Aug. 10, 2023

Related U.S. Application Data
(60) Provisional application No. 63/370,983, filed on Aug. 10, 2022.

(51) Int. Cl. A61B 5/00; H02N 1/04; A61B 5/024

(52) U.S. Cl. CPC A61B 5/6824; H02N 1/04; A61B 5/02438

(57) ABSTRACT

A self-powered system for continuous real-time physiological signal monitoring. The system may comprise a charging component configured to generate power from movement of the user, and one or more pressure sensors applied to a user, operatively coupled to the charging component, configured to measure one or more physiological signals of the user and output one or more capacitance values. The charging component may be further configured to power the one or more pressure sensors, and the one or more pressure sensors may comprise MXene. The charging component may comprise an MXene-based triboelectric nanogenerator.

### CROSS-REFERENCES TO RELATED APPLICATIONS

This application is a non-provisional and claims benefit of U.S. Provisional Application No. 63/370,983 filed Aug. 10, 2022, the specification of which is incorporated herein in its entirety by reference.

### FIELD OF THE INVENTION

The present invention is directed to wearable self-charging MXene-based devices for physiological signal measurement.

### BACKGROUND OF THE INVENTION

The development of wearable and flexible sensing devices has dramatically broadened the scope of personalized health monitoring. They offer a facile non-invasive approach to extracting real-time physiological data essential for health monitoring. Over the past decade, extensive studies have been carried out to fabricate wearable sensing devices with high sensitivity for the precise and accessible collection of vital signals. For instance, recently a stretchable vertical graphene network for respiration monitoring, a multifunctional 3D printed CNT-based pressure sensor to record pulses and vocal vibration, a wearable sensor patch to monitor electrocardiography, glucose, and temperature, an affordable sensor for pH monitoring, and an ultrasensitive aptamer-antibody cortisol sensor to monitor the stress state have been developed by different groups. However, most of such devices rely on rigid battery packs. Although flexible batteries enable conformal contact with the skin, limitations include charging requirements, replacement issues, and potential security risks. Thus, integrated sensing systems with sustainable power remain to be achieved. Though energy harvesting approaches such as photovoltaics, thermoelectric generators, and biofuel cells enable direct electricity acquisition from surroundings, their operation is restricted by external conditions, including temperature, light, and auxiliary catalysts. Therefore, more active and less environmentally dependent acquisition approaches could satisfy the requirements of mobility, wearability, and integrability.

Triboelectricity refers to the electrification of materials due to friction or frequent contact with another material. By collecting charges generated on material surfaces, triboelectric nanogenerators (TENGs) convert mechanical energy from human motion into electricity without previously mentioned restrictions (e.g., temperature, light). Due to their simple structure, low cost, universal availability, and high conversion efficiency, TENGs have broad applications and potential. For instance, in 2019, Bhaskar et al. introduced a polyaniline-based wearable TENG-based sensing system, which took advantage of the generated triboelectric output variation caused by external stimulus and used TENG directly as a pressure sensor. However, such sensors showed limited sensitivity without a micro-engineered active sensing layer or a portable data acquisition unit. In 2020, Gao et al. developed a triboelectrically driven system powering sweat biosensors for biomarker monitoring. However, their system was limited to recording discrete data points, minutes apart, and incapable of continuous monitoring of transient physiological biosignals. Thus, accurate, efficient, continuous, and real-time recording of transient physiological signals remains to be achieved.

The innovation of three-dimensional (3D) printing has revolutionized conventional fabrication and manufacturing technology over the past decade. As 3D printing matures, its scope is expanding from simple mechanical structures to functional devices. The 3D printing technique provides easy tailoring and fast prototyping of the customized design, which has a specific requirement and is complicated to fabricate by conventional manners, such as machining or casting methods. The innovation of 3D printing has revolutionized conventional fabrication and manufacturing technology over the past decade. Utilizing 3D-printing technology allows researchers with limited production abilities and industry experts to execute fast prototyping of designs that are complicated to manufacture with conventional processes such as machining or casting. Moreover, the 3D printing technique usually has a higher repeatable performance in the fabrication compared to the other manufacturing technologies, including soft lithography and infrared laser micro-machining. The advantages of this technique and the growing demand for internet-of-things and wireless electronics accelerate the development of 3D printed devices with mass customization, lightweight, and low material waste.

### BRIEF SUMMARY OF THE INVENTION

It is an objective of the present invention to provide systems that allow for wearable self-charging MXene-based devices for physiological signal measurement, and for the other sensing applications, as specified in the independent claims. Embodiments of the invention are given in the dependent claims. Embodiments of the present invention can be freely combined if they are not mutually exclusive.

The present invention features a novel, self-powered, “all-in-one”, MXene-based, 3D-printed, and integrated wearable sensing system for on-demand, continuous, and real-time vital signal monitoring. It can also be used for other battery-free sensing applications such as force sensing, pressure sensing, motion sensing, and more. The system includes highly efficient TENGs, highly sensitive pressure sensors, and multifunctional circuitry. MXene, a two-dimensional (2D) transition material with distinctive triboelectric properties, outstanding conductivity, and mechanical flexibility has been applied to the triboelectrification layers for the TENG and the conducting layers for the pressure sensors. Sensors comprising MXene may be used for simultaneous power generation and sensing purposes. Moreover, its shear-thinning viscoelastic property provides opportunities for additive 3D manufacturing in various devices. The 3D-printed, wearable, MXene-based, self-powered physiological signal sensing system (MSP²S³) exhibits an output power of ˜816.6 mW m⁻² for its TENGs, the sensitivity of 6.03 kPa⁻¹, a low detection limit of 9 Pa, and a fast response time of 80 ms for its pressure sensors (FIG. 1A). This enables continuous, real-time, and on-demand radial artery pressure (RAP) waveform monitoring without any external power supply.

The present invention features a self-powered system for continuous real-time physiological signal monitoring. Additionally, the system of the present invention can sense parameters such as pressure, force, motion, and more. In some embodiments, the system may comprise a charging component configured to generate power from movement of the user, and one or more pressure sensors applied to a user, operatively coupled to the charging component, configured to measure one or more physiological signals of the user and output one or more capacitance values. The charging component may be further configured to power the one or more pressure sensors, and the one or more pressure sensors may comprise MXene. The charging component may be further configured to perform sensing while generating power as well, by incorporating MXene. The charging component may comprise an MXene-based triboelectric nanogenerator. The system may further comprise a wearable component configured to attach to a user. In some embodiments, the system may further comprise a capacitance-to-digital converter (CDC) operatively coupled to the one or more pressure sensors and the charging component, configured to convert the one or more capacitance values into one or more digital signals. The system may further comprise a communication component operatively coupled to the CDC and an external source, configured to transmit the one or more digital signals to the external source. In some embodiments, the communication component may be configured to be charged by an external charging source.

One of the unique and inventive technical features of the present invention is the implementation of MXene as a material for the one or more pressure sensors. MXene can be used in nanogenerators or sensors, or both. Without wishing to limit the invention to any theory or mechanism, it is believed that the technical feature of the present invention advantageously provides for a physiological signal measuring system capable of greater electrical characteristics and mechanical stability than polytetrafluoroethylene (PTFE). None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a schematic of the MXene-based self-powered physiological sensing system (MSP²S³), which comprises an MXene-based TENG (M-TENG); an MXene-based pressure sensor (M-PS), both fabricated via additive manufacturing (3D printing) using home-modified MXene ink; a power-management circuitry; an energy-storing circuitry; and data collecting, and wireless data/power transmitting modules.

FIG. 1B shows a system-level block diagram illustrating the MSP²S³'s different modules in modes one and two.

FIGS. 2A-2I show characterizations for extrusion-printable MXene inks. FIG. 2A shows a schematic of the chemical structure of 2D Ti₃C₂Tx MXene. FIG. 2B shows an optical image of MXene ink, showing its gel-like viscous nature. FIG. 2C shows XRD patterns of the synthesized MXene (Ti₃C₂Tx) and MAX phase (Ti₃AlC₂) powder; the inset shows the 2.38° downshift of the (002) peak, indicating the intercalation of ions into the interlayer. FIG. 2D shows the FTIR pattern of the synthesized MXene. FIG. 2E shows a side-view SEM image of an MXene film showing the stack layers of nanosheets. FIG. 2F shows a top-view SEM image of the large flake MXene of the 3D printable ink showing surface morphology and typical flake sizes. FIG. 2G shows the lateral size distribution of large MXene flakes. The sizes of 200 flakes are counted from 5 different 150 μm×100 μm rectangles of a silicon wafer deposited with diluted MXene dispersion of large flakes. FIG. 2H shows viscosity plotted as a function of shear rate for the 3D printable MXene ink. The data followed the Ostwald-de Waele power law:η=kγⁿ⁻¹, where k and n are the flow index and shear-thinning power index, respectively. FIG. 2I shows rheological properties of 3D printable MXene ink with the storage and loss modulus plotted as a function of strain.

FIG. 3A shows a working mechanism of power generation from M-TENG in contact-separation mode.

FIG. 3B shows a working mechanism of power generation from the single-electrode M-TENG in bend-release mode.

FIG. 3C shows a simulation result for the potential difference over the change in the separation distance.

FIG. 3D shows the output open circuit voltage of the M-TENG on various contact-separation frequencies when subjected to 19.9N.

FIG. 3E shows the output open circuit voltage of the M-TENG on various applied forces when subjected to the operation frequency of f=8 Hz.

FIG. 3F shows the output voltage and current of the M-TENG generated on contact-separation frequency of f=8 Hz with 19.9 N vertical force as a function of the load resistance.

FIG. 3G shows the instantiated output power of the M-TENG as a function of the load resistance.

FIG. 3H shows a schematic of the M-TENG with a rectifier circuit.

FIG. 3I shows the capacitor voltage characteristic curves for various capacitors charged through the M-TENG.

FIG. 3J shows the voltage output characteristics of the single electrode M-TENG in response to bending at different bending angles of 30°, 60°, and 90° using a linear motor. A voltage signal of the M-TENG in response to the continuous bend of the finger in FIG. 3K, and the elbow in FIG. 3L.

FIGS. 4A-4F show the characterization of the M-pressure sensor. FIG. 4A shows a schematic of the M-PS. FIG. 4B shows a characteristic curve for the M-pressure sensor, showing the relative change in capacitance for different external pressures. FIG. 4C shows the response time and FIG. 4D shows the relaxation time of the M-PS. FIG. 4E shows the minute pressure response from applying droplets of water. FIG. 4F shows the actual pulse signal collected from the M-pressure sensor and the enlarged view for one specific pulse shows the three typical peaks of the wrist pulse.

FIGS. 5A-5D show an application for wearable MSP²S³ for detecting the wrist pulse as a representative physiological signal using only biomechanical energy. FIG. 5A shows a circuit diagram for the MSP²S³ including the M-TENGs, power management circuitry, energy storing circuitry, data collection, and wireless data/power transmission modules. FIG. 5B shows an optical image of the wearable MSP²S³. The inset “on” and “off” status of the LED, respectively, represent the valley and peak of the pulse signal detected by the MSP²S³ in mode one. FIG. 5C shows the enlarged view for one specific pulse, showing the typical three peaks of a single wrist RAP pulse. FIG. 5D shows the real-time pulse data monitored using MSP²S³ in mode two, displayed in the customized App.

### DETAILED DESCRIPTION OF THE INVENTION

The term “MXene” is defined herein as a two-dimensional inorganic compound comprised of atomically thin layers of transition metal carbides, nitrides, or carbonitrides.

The term “triboelectric nanogenerator” is defined herein as an energy harvesting device that converts mechanical energy into electricity using the triboelectric effect. The triboelectric effect (also known as triboelectricity, triboelectric charging, triboelectrification, or tribocharging) describes electric charge transfer between two objects when they contact or slide against each other.

The term “capacitance” is defined herein as the ratio of the change in an electric charge in a system to the corresponding change in its electric potential.

The term “near-field communication chip” is defined herein as a silicon component or Integrated Circuit (IC) that enables short-range, wireless communication between two devices.

The present invention features a self-powered system (100) for continuous real-time physiological signal monitoring. In some embodiments, the system (100) may comprise a charging component (110) configured to generate power from the movement of the user, and one or more pressure sensors (120) applied to a user, operatively coupled to the charging component (110), configured to measure one or more physiological signals of the user and output one or more capacitance values. The charging component (110) may be further configured to power the one or more pressure sensors (120), and the one or more pressure sensors (120) may comprise MXene.

In some embodiments, the charging component (110) may comprise a MXene-based triboelectric nanogenerator. The triboelectric nanogenerator may comprise MXene, gold film, and Styrene-ethylene-butylene-styrene stacked in layers in any configuration. In some embodiments, the system (100) may further comprise a wearable component (130) configured to attach to a user. The wearable component (130) may be configured to attach to a wrist, arm, leg, stomach, chest, neck, or any portion of exposed skin of the user. In some embodiments, the system (100) may further comprise a capacitance-to-digital converter (CDC) (140) operatively coupled to the one or more pressure sensors (120) and the charging component (110), configured to convert the one or more capacitance values into one or more digital signals.

In some embodiments, the system (100) may further comprise a communication component (150) operatively coupled to the CDC (140) and an external source (170), configured to transmit the one or more digital signals to the external source (170). In some embodiments, the communication component (150) may comprise a microcontroller unit (MCU) and a near-field communication (NFC) chip further configured to be charged by an external charging source (180). In some embodiments, the external charging source (180) may comprise a smart device, a wireless battery, or any device capable of wirelessly transmitting an electric charge. In some embodiments, the external source (170) may comprise a smart device, a personal computing device, a cloud server, a physical server, or any device capable of receiving digital information. In some embodiments, the system (100) may further comprise one or more light-emitting diodes (LEDs) (160) operatively coupled to the one or more pressure sensors (120) and the charging component (110), configured to actuate in response to the movement of the user.

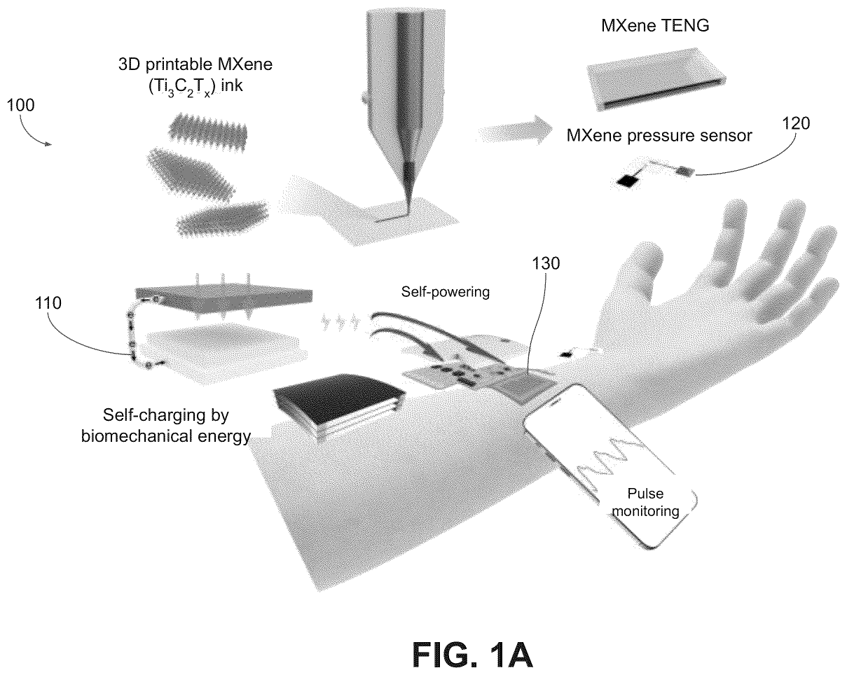

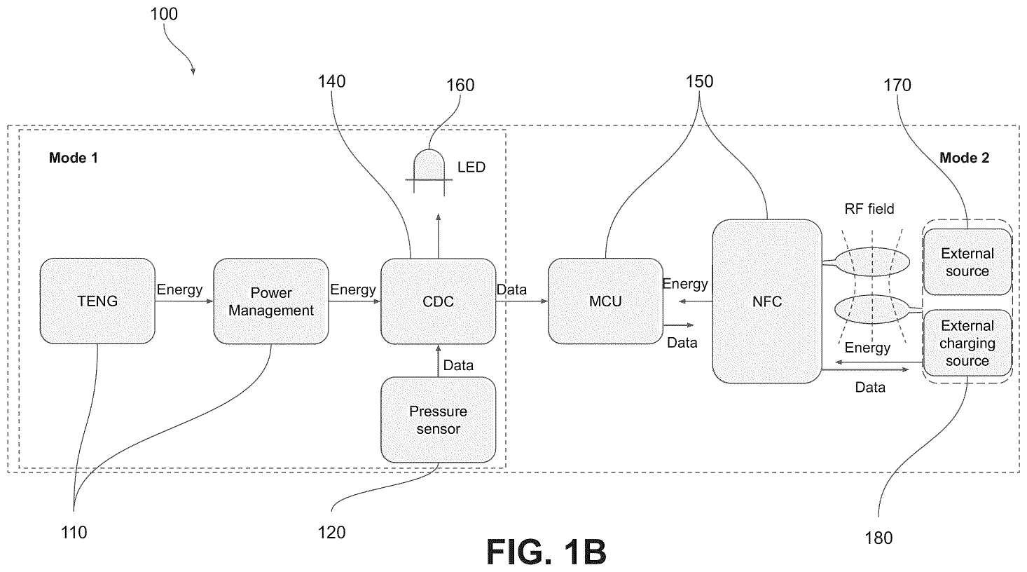

The MSP²S³ is a two-mode system (FIG. 1B). In mode one, the system is entirely self-powered by the TENG units, measuring RAP peaks from the wrist continuously in real-time. Specifically, the MXene-based TENG units harvest the mechanical energy from the user's finger tapping, powering an embedded capacitance-to-digital-converter (CDC) chip, the MXene-based pressure sensors, and LEDs. The CDC chip communicates with pressure sensors and the LEDs to visualize the valleys and peaks of the measured RAP waveforms with the “on” and “off” states, respectively (one flash per pulse).

In mode two, the TENGs still power the CDC chip and pressure sensors, while a smartphone wirelessly powers a near field communication (NFC) chip and a microcontroller unit (MCU), enabling wireless power and data transmission. The MCU extracts the measured RAP data directly from the CDC chip, and the NFC chip transmits it to the phone for display and storage. A custom AndroidTM App decodes the data and plots the RAP waveforms in real-time. This is the first fully integrated, triboelectrically-driven-self-powered, battery-free, wireless, and MXene-based wearable sensing system for continuous real-time physiological signal monitoring.

The physiological signal measured by the system of the present invention may comprise heartbeat rate, respiratory rate, skin conductance, muscle current, skin temperature, blood pressure, internal temperature, or any other physiological signal able to be measured through the skin of a user by pressure.

The microcontroller unit (MCU) of the present invention may comprise a processor configured to execute computer-readable instructions and a memory component comprising computer-readable instructions for accepting the one or more digital signals from the CDC and transmitting the one or more digital signals to the external source.

In some embodiments, the system as a whole may be 0.5 to 2 inches by 0.5 to 2 inches in area or smaller. The system may comprise 1 to 6 pressure sensors. The system may comprise nanogenerators that serve the dual purpose of power generation and sensing simultaneously.

### EXAMPLE

The following is a non-limiting example of the present invention. It is to be understood that said example is not intended to limit the present invention in any way. Equivalents or substitutes are within the scope of the present invention.

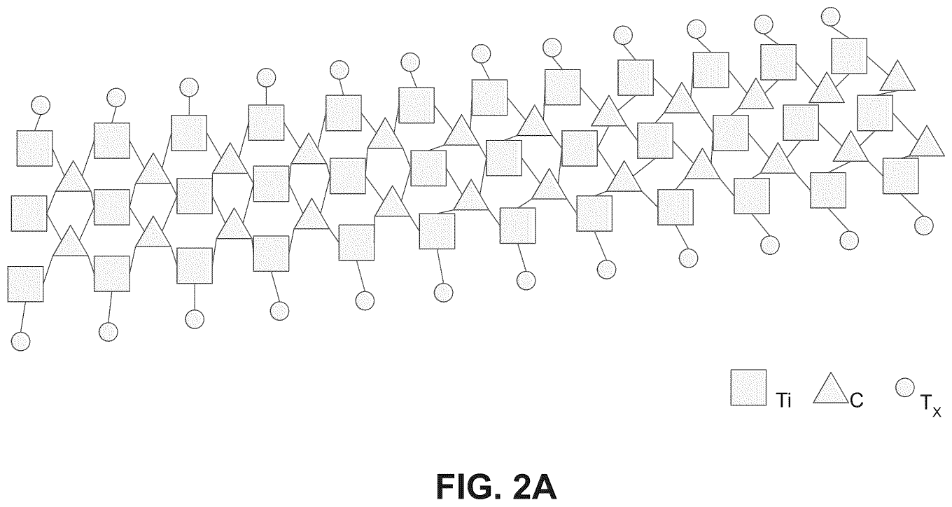

MXene, a recently emerging 2D material, has received extensive interest for its excellent electrical characteristics and mechanical stability. Its carbon backbone enables electricity passage and out-plane flexibility (FIG. 2A). In addition, its abundant surficial termination groups (hydroxyl, oxygen, and fluorine) make MXene triboelectric similar or even more negative than the commonly used triboelectrically negative material-polytetrafluoroethylene (PTFE) in triboelectric series. As a result, MXene is an ideal material for flexible triboelectric layers and conductive electrodes.

Ti₃C₂Tx MXene ink was synthesized using the minimally intensive layer delamination (MILD) method by selective etching of the aluminum layers from the Ti₃AlC₂ MAX phase and delaminated into 2D monolayer sheets using intercalation agents such as metal ions or organic molecules. A viscoelastic gel-like black sediment was obtained after synthesis and purification, with several material characterizations (FIG. 2B). First, the phase composition and crystal structure analyses were conducted using an X-ray diffractometer (XRD), and Ti₃AlC₂ and the synthesized Ti₃C₂Tx patterns were compared. As illustrated in FIG. 2C, the characteristic peaks of Ti₃AlC₂ in the range of 33°-43° of Ti₃AlC₂ eventually disappeared, indicating completed etching. In addition, the (002) peak of the synthesized Ti₃C₂Tx MXene shifted toward a smaller 2θ angle by 2.38°, indicating the expansion of the c-lattice parameter. This downshift was because of the intercalation of ions and/or small molecules into the interlayer of MXene, indicating delamination of the 2D sheets. Next, Fourier transform infrared spectroscopy (FTIR) was performed for chemical bonding identification. The spectrum of synthesized Ti₃C₂Tx reflecting the chemical composition of MXene is shown in FIG. 2D. Peaks at 3301 and 1631 cm⁻¹ indicated the presence of strong hydrogen bonding (—OH), and the peak at 583 cm⁻¹ was attributed to the deformation vibration of the Ti—O bond. In addition, the morphology of the extrusion-based 3D-printed MXene film was measured by cross-sectional SEM imaging. A well-percolated nanosheet network was obtained, showing the 2D sheet delamination and the natural flake alignment via the 3D extrusion printing process (FIG. 2E).

Various factors affecting the electrical and mechanical properties of MXene include particle flake size, defects on the MXene flakes due to sonication, voids in the film due to the irregular stacking of the flake, and flake alignment. Generally, the larger the MXene flakes, the greater the electrical conductivity. To maximize the electrical performance of the 3D-printable MXene ink, two refinements were made: i) the size of the raw MAX phase powers was selected to relate directly to the flake size after etching; and ii) vigorous sonication was avoided to ensure minimal damage to the Ti₃C₂Tx flakes, instead of employing metal ion intercalation and moderate magnetic bar stirring. Flake morphology and size distribution for the refined large flakes (FIGS. 2F-2G) and broken small flakes were characterized by top-view SEM. The electrical conductivity performance was also measured under different refinement processes. The refined large flakes and broken small flakes exhibited a mean lateral size (<I>) of 4.12 μm and 0.29 μm, respectively, indicating flake elimination in the submicron scale. The electrical conductivity performance enhancement under different refinement stages was achieved. MILD synthesis without flake size modification yielded ink conductivity of 1535±396 S/cm. With the selection of MAX phase particles >10 μm, higher conductivity of 3616±598 S/cm was obtained. The elimination of broken flakes under the micro-scale during purification further increased the ink conductivity to 5096±332 S/cm. The lower conductivity noted in unmodified ink is attributed to the presence of more inter-particle contacts in conductive paths resulting in a higher scattering rate of electrons at boundaries with the presence of small particles. Thus, flake size refinement remarkably enhanced the electrical performance of the 3D-printable MXene ink, compared to previous reports.

To determine the suitability of MXene ink for additive manufacturing, viscosity must first be characterized. It describes the resistance to flow as a relationship between stress and deformation rate. Though extrusion at a high shear rate requires low viscosity, high viscosity is necessary to retain the printed shape. This material performance characteristic called shear-thinning behavior, can be characterized using the Ostwald-de Waele power-law viscoelastic model (Eq. (1)), with the power index n between 0 and 1 (0<n<1):

μ=kγⁿ⁻¹ (1)

where μ is the viscosity, γ is the shear rate, n is the power index, and k is the flow index.

As shown in FIG. 2H, after fitting the experimental data, MXene ink at 4 wt % exhibited a typical shear-thinning behavior with n=0.07. Additionally, as shown in FIG. 2I, by increasing the shear rate, the dominant modulus of the ink changes from storage modulus to loss modulus that is favored for the extrusion process. Viscosity can be further modified by tuning the ink concentration. The presence of the hydroxyl functional group in the MXene flakes favors mixing with water. Homogeneous mixtures with different viscosities can be obtained by managing the solvent ratio (i.e., 4 wt %, 4.5 wt %, and 5 wt %, FIG. 2H). The power index (n) and flow index (k) followed the governing equation of n=1−0.57594(C)^{0.3156} and k=−172 C²+1980 C−5010, respectively, depending on the ink concentration, allowing adjustment of viscosity to different extrusion systems to achieve a high printing resolution of ˜100 μm.

As described in the previous section, MXene has a highly triboelectric negative surface due to the presence of fluorine and oxygen-containing terminal functional groups. When MXene couples with materials with opposing triboelectric surfaces, triboelectric charges are generated from the contact and separation cycles. For wearable devices, a skin-like flexible substrate that can accommodate deformation during body movement should be used. Styrene-ethylene-butylene-styrene (SEBS) is ideal for the electric-skin, because it has a high stretching limit of up to 400%, accommodating many different deformations, and its triboelectric positive property allows electricity generation upon pairing with MXene (triboelectric negative).

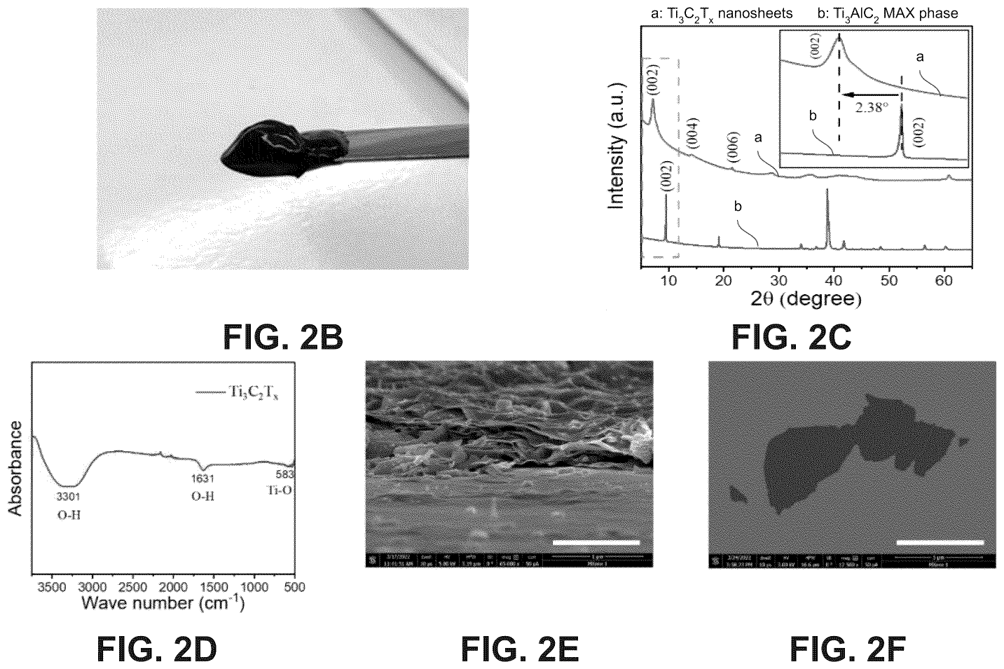

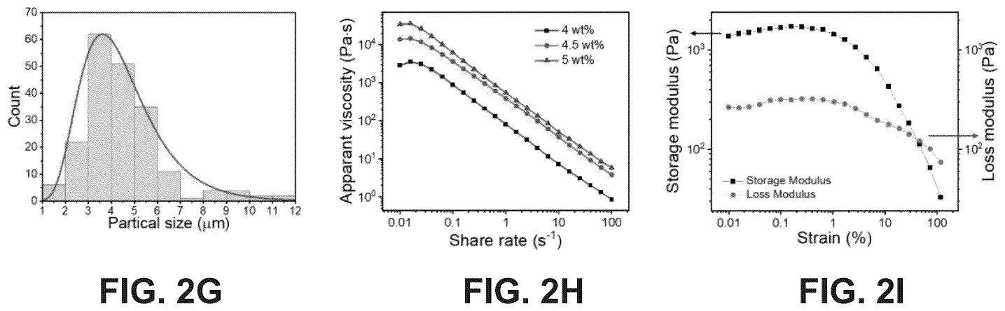

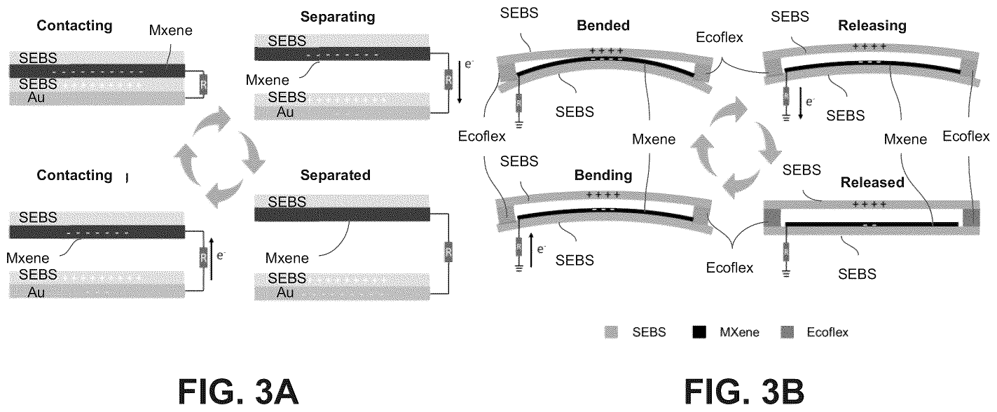

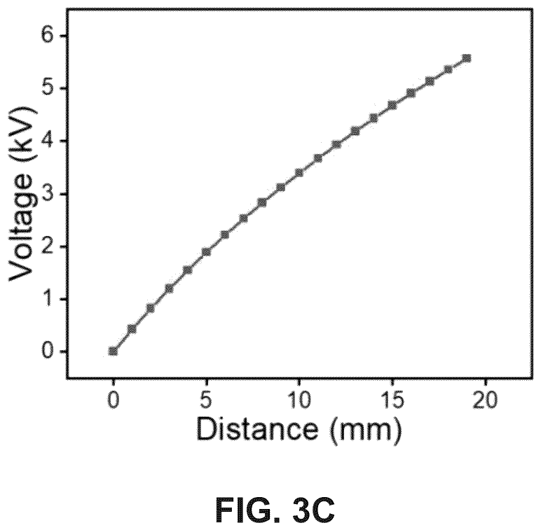

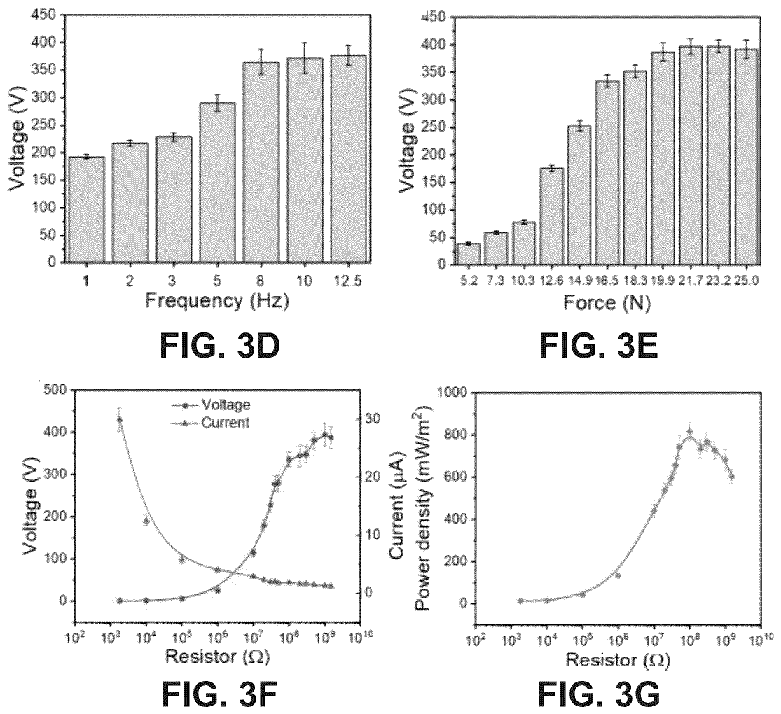

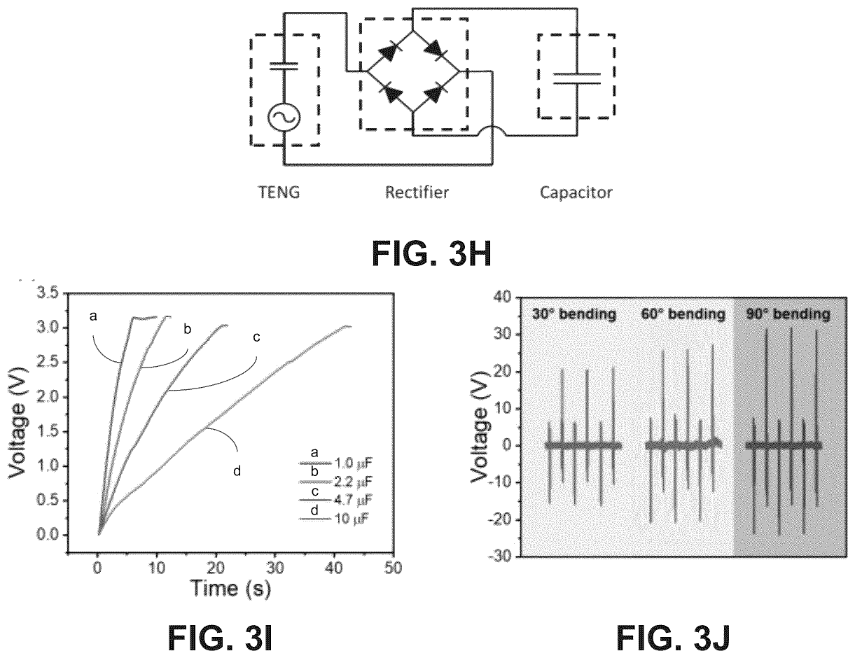

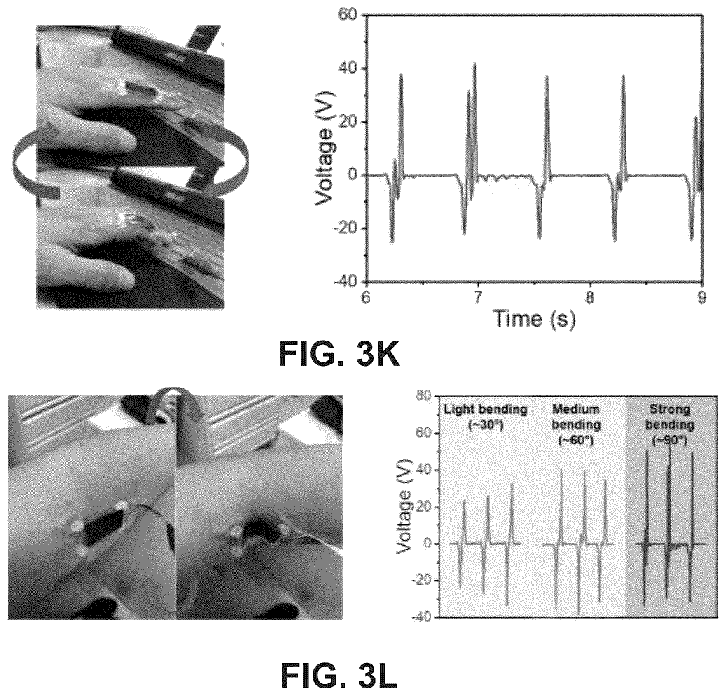

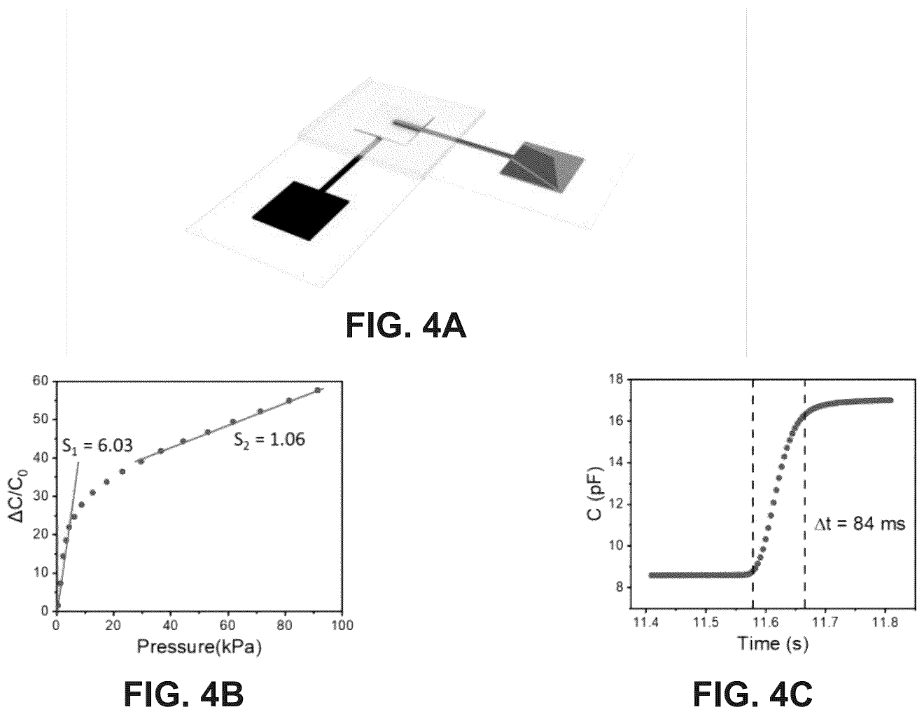

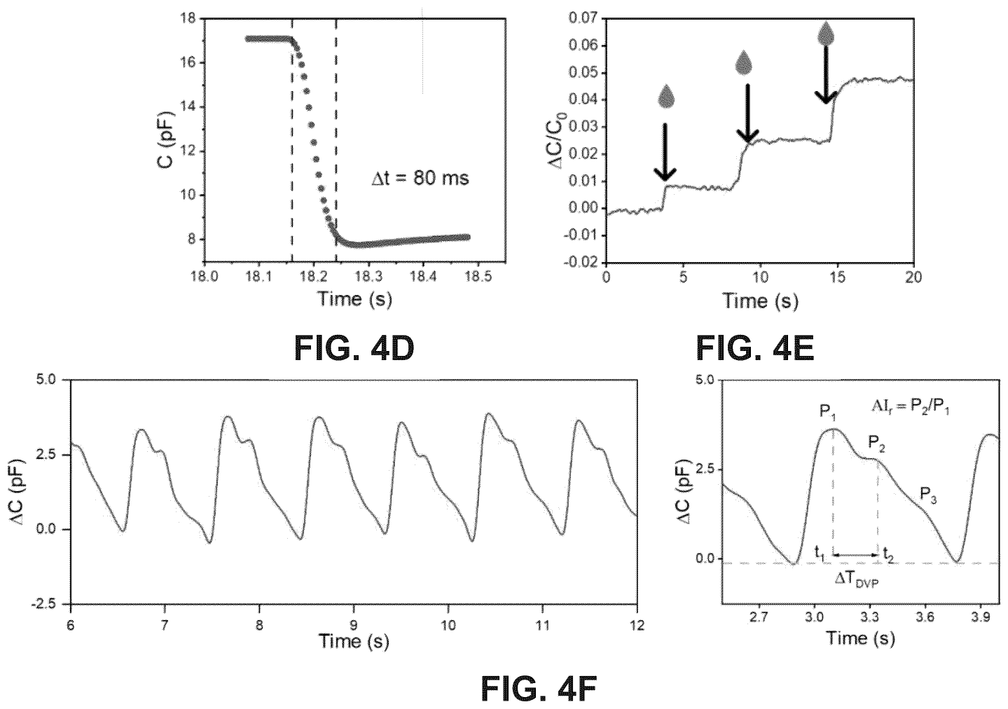

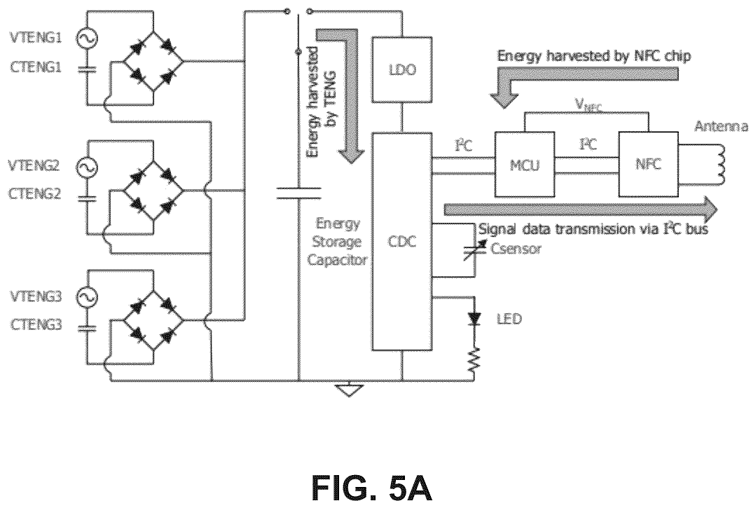

The working mechanism of the M-TENG in contact-separation mode and the single electrode M-TENG in bend-release mode are illustrated in FIGS. 3A-3B, respectively. In the initial stage, the triboelectric pair layers are separated and maintained in a neutral condition with no additional charges, in the contact-separation mode. When an external force is applied, triboelectric negative MXene is brought into contact with the triboelectric-positive SEBS, and electrons are transferred from SEBS to MXene. Upon separation, electrons on the conductive MXene film flow to the other electrode due to the edge electric field leakage effect, with the positive charges remaining on the surface of SEBS. Electrons then flow back to the MXene film to balance the positive triboelectric charges on SEBS as the two films are brought into contact again. Therefore, in the case of continuous contact and separation, alternating electrical signals are generated. The electrostatic potential distribution increases according to the separation distance.

The power conversion performance of the energy harvester was evaluated. The contact-separation movement was controlled using a linear motor, and the open-circuit voltage (Voc) profiles of the M-TENG at different working frequencies and applied forces were examined. As shown in FIG. 3C, the operation frequency gradually increased from 1 Hz to 12.5 Hz at a controlled applied force of 19.9 N. The output performance of the M-TENG first increased, and then settled at the frequency of 8 Hz, which can be attributed to surface charge accumulation on the triboelectric layer surface. However, increasing the frequency above 8 Hz prohibited neutralization of the accumulated charges. Similarly, as shown in FIG. 3E, the effect of the applied force was studied by gradually increasing the applied force from 5.2 N to 25.0 N at an operational frequency of 8 Hz. Greater force (i.e., 19.9 N) increased the output power, which was attributed to maximizing frictional contact on the MXene and SEBS surfaces. The optimum working conditions of 8 Hz and 19.9 N were maintained in subsequent experiments to determine the maximum output power for the fabricated M-TENG device.

To investigate the M-TENG's maximum power output, its voltage, current, peak power, and peak power density performances were measured under a series of external loads. The output voltage increased with increasing external load, whereas the current followed a decreasing trend (FIG. 3F). The instantaneous power density of the M-TENG reached a maximum of 816.6 mW m−2 7 under a load resistance of 10 8 8 Ω (FIG. 3G). Next, the charging efficiency performance for different energy-storage capacitance values was characterized. As shown in FIG. 3H, a full-wave rectifier was added to the circuit to convert the alternating current generated from the mechanical energy by M-TENG into direct current, which was stored in the capacitor. Charging efficiency was examined by quantifying the charging time of different capacitors. Capacitors of 1.0, 2.2, 4.7, and 10 μF were charged by finger tapping at the frequency of 3 Hz (convenient for one to perform). As seen in FIG. 3I, the storage voltage reached 3 V within 5 s for the 1 μF capacitor, and the 10 μF capacitor was charged to the same voltage within 50 s, indicating the reasonable charging capability of the M-TENG.

Additionally, the single electrode M-TENG is characterized, as shown in FIGS. 3J-3L. Given its thinness, the single-electrode M-TENG can be placed at locations with more significant curvature changes and thus harvest energy. As shown in FIG. 3J, with the increasing bending angle (30°, 60°, and 90°), the voltage output of the M-TENG increased gradually. This phenomenon may be the product of the expanded contact area between the MXene and the SEBS, further promoting triboelectric charge generation. When attached to fingers or the inner elbow, as shown in FIGS. 3K-3L, the single electrode M-TENG harvested electricity from the bending of the arms and figures, and the energy increased with larger bending angles. However, according to the characterization experiments, the M-TENG in contact-separation mode produced a higher power intensity compared to the single-electrode M-TENG in bend-release mode. Subsequently, the M-TENG was implemented in the contact-separation mode in MSP₂S₃.

Although the TENGs can be used as motion detection sensors, their lack of sensitivity hinders the detection of subtle physiological signals such as wrist pulses, which contain a wealth of health information. Therefore, highly sensitive sensors had to be integrated into the self-powering systems. Among sensors developed to date, the capacitive-based pressure sensor offered high sensitivity, low power consumption, simple design, high immunity to environmental noise, and relatively fast response time to external stimuli.

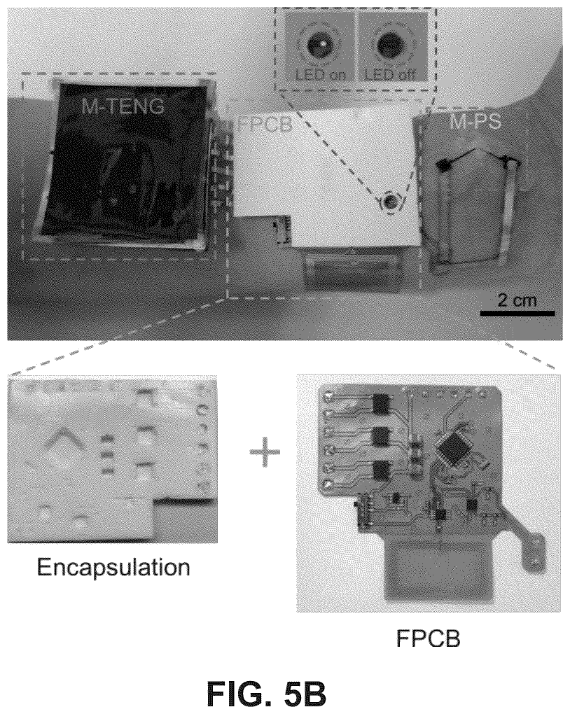

The structure of the MXene-based capacitive pressure sensor is illustrated in FIG. 4A. Epidermal SEBS was chosen as the sensor substrate for providing conformal contact to human skin, thus ensuring minimum signal attenuation and avoiding discomfort due to modulus mismatch with the skin. Conductive electrodes were fabricated via 3D-extrusion printing using MXene ink. The sandpaper-molded dielectric [EMI][TFSA]:PVDF layer was sandwiched between the two conducting MXene layers to enhance deformation under external pressure and further increase sensitivity. The sensitivity of the M-PS was characterized using a force gauge (applying external pressure) and an LCR meter (for reading capacitance changes) and quantified as the slope of its calibration curve (Eq. 2).

$$S = \frac{d ( Δ C / {C}_{0} )}{d ( Δ P )} \qquad ( 2 )$$

As shown in FIG. 4B, the sensitivity of the M-PS was measured in two regions: S1 (6.03 kPa−1 1) in the low-pressure region (0-4 kPa) and S2 (1.06 kPa−1 2) in the high-pressure region (4-100 kPa). In the low-pressure region, even slight applied pressure induced a large deformation in the dielectric material due to the highly porous sandpaper-imprinted structure, and the soft material property of [EMI][TFSA]: PVDF, which increased capacitance, yielding very high sensitivity. In the high-pressure region, the deformation of the micro-structured layer was almost saturated because of the reduction in porosity, increasing the stiffness of the polymer, thus sensitivity was compromised compared with the low-pressure region.

The response and relaxation times of the fabricated sensor were evaluated under the pressure of ˜1 kPa (FIGS. 4C-4D). A dynamic response time of 84 ms and a relaxation time of 80 ms were achieved; this suggests that the time for elastic recovery for [EMI][TFSA]: PVDF polymer is the same as that for compression, indicating negligible hysteresis. Moreover, the limit of detection (LOD) for minute pressure was characterized by investigating the sensor's response under the applied pressure of a water droplet (12 μL, ˜0.009 kPa). The sensor was fixed to a stage and connected to the measurement circuit of an LCR meter. After obtaining a stable baseline, water droplets were dispensed onto M-PS. As shown in FIG. 4E, M-PS exhibited high sensitivity to external ultra-low load by detecting a single water droplet (12 μL, ˜0.009 kPa). The change in the capacitance corresponding to the introduction of each droplet was identical, indicating the linear behavior of the M-PS in a low-pressure regime.

Besides its outstanding performance in the detection of minute pressure, the sensor's application in real-time pulse monitoring was also demonstrated. The sensor was attached to the wrist of a 28-year-old healthy male volunteer using double-sided medical tape, and the pulse signal was continuously recorded for 45 seconds. The real-time wrist pulse signal results obtained by the M-PS are shown in FIG. 4F. In the detailed characteristics of a single RAP waveform, an incident wave generated by blood flow (P₁), the late systolic shoulder (P₂), and another tiny reflection wave (P₃) originating from the lower body were clearly visible, demonstrating precise signal monitoring. Radial artery augmentation index and the associated time delay defined as AIᵣ=P₂/P₁ and ΔT_{DVP}=t₂−t₁, respectively, are commonly employed for estimating the physiological condition of the human cardiovascular system. The results revealed the two parameters AIᵣ and ΔT_{DVP} as 0.761 and 0.235 s, respectively, which were representative values for a healthy young man (0.695±0.163). The long-time monitoring result indicated that the pulse rate was 70, which is also within the expected range for a healthy young man (60-100 bpm). The results confirm that the proposed M-PS can identify subtle differences in the wrist pulse, provide useful information regarding the cardiovascular system, and affirm the potential in practical applications.

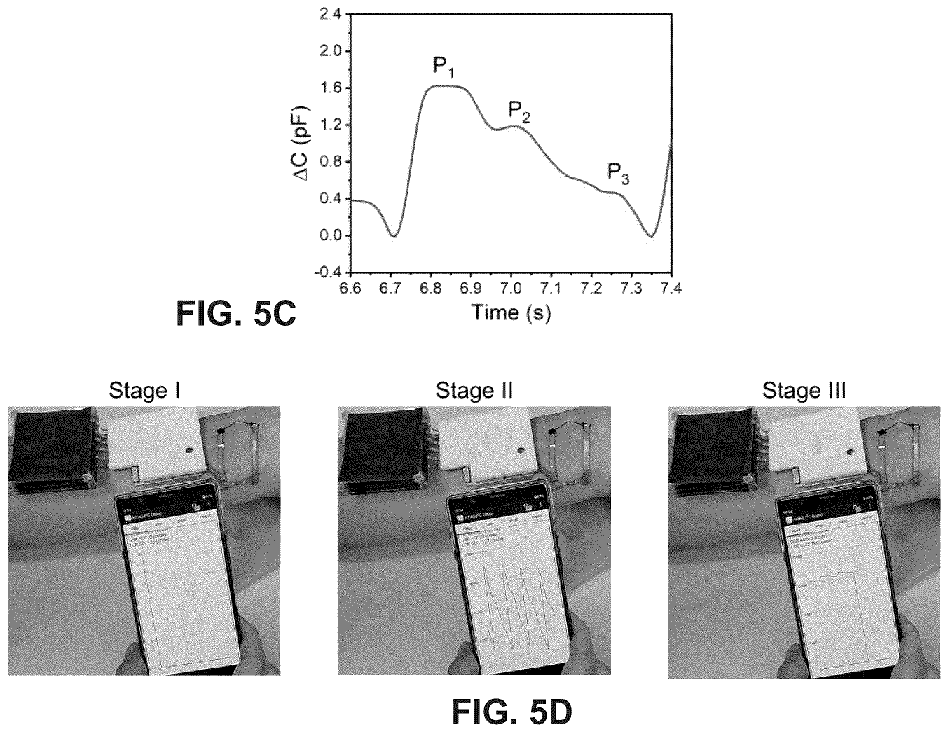

For the practical application of real-time and continuous physiological signal monitoring powered by biomechanical energy, the utility of the MSP²S³ was demonstrated. In terms of design, the structure of the self-powered MSP²S³ comprises i) three M-TENGs to produce power, ii) custom-designed circuitry on a flexible printed circuit board (FPCB) for power management, energy storage, and data collection, visualization, and wireless data transmission, iii) an M-PS for RAP measurements. As shown in the circuit diagram in FIG. 5A, biomechanical energy from finger tapping was first converted to electricity by the M-TENG. Next, the generated AC current was rectified to DC using full-wave rectifiers and stored in the capacitors on the FPCB. As described above, the MSP²S³ was designed to work in two modes. In mode one, the stored energy powered the CDC chip which communicated with the M-PS to measure RAPs in real-time and control an LED to flash at a rate indicating the measured pulse rate during the active data recording. In mode two, the detailed measured RAP waveforms were wirelessly transmitted to a smartphone through the NFC technology for further analysis and possible detection of abnormalities.

Here, to enhance the MSP²S³ performance, two main parameters needed to be considered: increasing the power generation efficiency and minimizing power consumption. For the first parameter, a more extensive panel area and multiple stacks of M-TENGs were employed. The charging performance of the M-TENGs at the frequency of ˜3 Hz with various numbers of parallel stacks was recorded. While for a single stack M-TENG, ˜252 seconds was needed to charge a capacitor at 3.4 V, this time was reduced to ˜180 seconds and 154 seconds in two-stack and three-stack M-TENGs, respectively. However, as more stacks of M-TENGs were incorporated, more MXene layers were necessary to attenuate under the same applied force, which did not lead to a linear improvement of the charging rate. For the second consideration, the power consumption at the system design level was reduced by adding a low-dropout regulator (LDO) to control power consumption and extend operation time per charging phase. This LDO maintained the operating current <50 μA to allow the CDC chip and LED to operate while avoiding power wastage.

Next, the utility of MSP²S³ for in-situ and the real-time RAP monitoring in mode one was demonstrated to show the RAP peak rates with the LED. The MSP²S³ was first attached to a volunteer's arm and set to the charging phase (FIG. 5B). The user was asked to start finger-tapping the M-TENGs to charge the storage capacitor, then switch the device to powering phase to power the CDC chip, M-PS sensor, and the LED. Subsequently, the LED began to flash, with “on” and “off” states representing the valleys and peaks of the RAP waveform, respectively (FIG. 5B).

The utility of MSP²S³ in mode two was also demonstrated. As explained before, the M-TENG still powers the CDC chip, M-PS sensor, and LED in this mode. However, the NFC chip and MCU were wirelessly powered by a phone to extract the measured RAP data directly from the CDC chip and wirelessly transmit the data to the phone for analysis and real-time plotting on a custom-made Android™ App. As shown in FIG. 5D, after the charging phase and making sure the phone was near to the NFC antenna for power and data transmission, a line with a value of zero first appeared on the App because the CDC chip was not still powered. By switching the MSP²S³ from the charging phase to the powering phase, it started to power the CDC chip and M-PS sensor to collect the RAP waves, while the NFC chip wirelessly transmitted the data to the phone for display. Eventually, the storage capacitors ran out of power, stopping power flow to the CDC chip and M-PS sensor. The NFC chip read and transmitted the null value again, as shown in FIG. 5D.

One should note that recharging (tapping) and powering phases can be repeated to measure RAP waves over any period of time. In addition, wirelessly transmitted RAP data can be further analyzed to extract more details about the waveforms. For instance, in RAP waves P₁, P₂, and P₃ in FIG. 5C, the two principal parameters of AIr and ΔTDVP were extracted as 0.725 and 0.20 s, respectively. Throughout this work, the RAP data collected by the MSP²S³ were always cross-validated with commercially available tools (e.g., an impedance spectroscope). One should note all sets of data were collected from healthy volunteers with no known cardiovascular disease problems in this work. Additionally, as a possible future development of the work, implementation of a fully automated MSP²S³ can be considered in which the devices can automatically switch between two different modes, leading to minimizing user involvement and potentially enabling continuous and real-time biosignals collection without interruptions.

In summary, a novel, flexible, wearable 3D-printed MXene-based self-powered and wireless sensing system has been developed for physiological signal monitoring. With seamless integration of M-TENG, M-PS, and multifunctional circuitry, power was generated from mechanical motion and efficiently applied for continuous and real-time RAP wave monitoring. MXene has a high triboelectric negative property and exhibits a high output power of 816.6 mW m⁻² when coupled with the SEBS in the M-TENG. Moreover, with modifications, the MXene demonstrated a threefold improvement in conductivity and tunable viscoelastic property favored for 3D printing. The M-PS of the proposed MSP²S³ system showed a high sensitivity of 6.03 kPa⁻¹ and a fast response time of 80 ms, enabling the capture of subtle changes in transient biosignals.

The utility of the MSP²S³ for on-demand, continuous, real-time, and self-powered RAP monitoring was demonstrated, as was the implementation of wireless power and RAP waveform transmission via NFC technology. This is the first fully integrated, triboelectrically-driven, self-powered, battery-free, wireless, MXene-based wearable sensing system for continuous and real-time physiological signal monitoring powered by human motion.

MAX phase particle selection: Ti₃AlC₂ MAX phase powder (2 g, <40 μm particle size, NANOCHEMAZONE™) was dispersed in 40 ml of water by magnetic stirring for 10 min. The mixture was left to stand for 3.5 minutes to separate MAX phase particles with a diameter larger than 10 μm by sedimentation. The relationship between the practical size and the sedimentation time is governed by the equation below:

$$v = \frac{2 ( {ρ}_{p} - {ρ}_{f} ) ℊ {R}^{2}}{9 μ}$$

Where v is the sedimentation speed, g is the gravitational acceleration, R is the radius of the spherical particle, ρₚ is the mass density of the MAX phase particles, ρ_{f} is the mass density of water, and μ is the dynamic viscosity of water. After the sedimentation process, the top supernatant containing suspended small MAX phase particles was decanted from the sediment, and the sediment was dispersed in 40 ml deionized (DI) water again for another cycle. The entire process was repeated three times with the same conditions to thoroughly remove small particles. The collected sediment was dried under vacuum at room temperature (25° C.) for 12 h before being used for synthesis.

1.6 g lithium fluoride (LIF, 99%, Sigma Aldrich™) was dissolved in 20 ml of 9 M hydrochloric acid (HCl) by magnetic stirring for 10 min. 1 g of size-selected MAX phase powder was then gradually added to the pre-mixed etching solution over 10 minutes. The MAX phase etching process was carried out at 50° C. for 30 h. After the reaction was completed, the resulting dispersion was washed with DI water by repeated centrifugation at 2,700 rpm (1,345 rcf) for 5 min per cycle until self-delamination occurred at a supernatant pH of ˜6. The self-delaminated MXene flakes were then collected by centrifugation at 1,180 rpm (247 rcf) for 30 min. The dark green supernatant was collected and further centrifuged at 3,500 rpm (2,223 rcf) for 20 min; the sediment containing large MXene flakes was collected for use as ink for extrusion 3D printing without any other process.

Viscosity-tunable ink was achieved by adding and evaporating the water content in the ink mixture, and the weight was measured at each stage to achieve different ink concentrations (4, 4.5, and 5 wt %). The flexible MXene film was directly printed using a commercial 3D extrusion printer (Incredible+™, Cellink Inc.™) on the styrene-ethylene-butylene-styrene (SEBS) substrate. The as-printed film was further annealed at 80° C. for 30 min.

MXene film was printed on the flexible SEBS substrate. The conductive (copper foil/gold-coated film) trace was connected to the MXene film with silver epoxy, then encapsulated and protected by the Ecoflex™ 00-30 (Smooth-On™, PA). 4 PDMS spacers (2 mm×2 mm×4 mm) were attached to the corners of the SEBS substrate with adhesive gels, and the other piece of the SEBS layer was attached to the top of the spacer with the same gel, and the gel was cured at room temperature for 24 hours.

Wearable M-PS was made starting from the MXene printing on the SEBS substrate. The dielectric layer was formed by spin coating intronic material [EMI][TFSA]: PVDF onto 200-grit sandpaper and annealing at 100° C. for 30 minutes. The structured dielectric was peeled away from the sandpaper mold and sandwiched between the MXene/SEBS conducting panel, forming the capacitive pressure sensor.

The FPCB comprised a microcontroller (ATmega328P, Microchip™), an NFC transducer (NT3H2111, NXP™), a CDC chip (AD7156, Analog Devices™), and some passive components. All the Integrated Circuits (ICs) and passive components are in small packages and sizes to increase compactness. A low-temperature solder paste (SMDLTLFPT5, CHIPQUIK™) was brushed onto the pads using a stencil. ICs and components were placed on the solder paste by hand and soldered by the reflow process. A customized Android™ app was developed to wirelessly communicate with the FPCB and analyze the data.

A field emission electron microscope (SEM, Hitachi™ 4700) was used to study the powder morphology and flake size distribution. MXene powder samples were prepared by vacuum filtration, and the flake samples were prepared by ink dilution and drop cast onto the silicon wafer. The flake size distribution was measured in 200 flakes out of 5 different SEM images (150 μm×100 μm for the large flake samples, and 1.8 μm×1.2 μm for the small flake samples) from the four corners and the center of the wafer. X-ray diffraction (XRD) patterns of the MAX phase and synthesized MXene were obtained using an X-ray Diffractometer (Rigaku Smartlab), equipped with Cu Kα radiation (40 kV, 44 mA) with an X-ray wavelength (λ) of 1.54 Å. Fourier transform infrared (FTIR) spectroscopy was used to characterize the functional groups on the surface of the synthesized MXene. Electrical conductivity measurements of the MXene ink during different treatments were conducted by using an LCR meter (GW Instek LCR-819), and the value was calculated from the obtained resistance and the filament geometry from the 3D printed patterns (n=5).

$$σ = \frac{l}{w \times t \times {R}_{0}}$$

Where w and t are the width and thickness of the printed line structure, respectively, I is the length, R₀ is the sample resistance, and a is the sample conductivity. A linear motor (PS01-23X80R, Linmot) was used for frequency and force control during the M-TENG characterization. The open-circuit voltage and short-circuit current were recorded by using a potentiostat (Versastat 3). The output power under different external loads was calculated from the corresponding voltage and resistance. The M-PS sensitivity characterization was performed using a force gauge connected to the LCR meter, the force was applied from 0 N to 22.4 N, and the force was further converted to pressure (P=F/A). The response time, relaxation time, and in-situ characterization of the pressure sensor were measured by an impedance spectroscope (HF2IS, Zurich instrument) under the operation frequency of 100 kHz, and voltage of 1 V with the sampling rate of 225 samples/sec. A custom-made MATLAB code was used to process the impedance data obtained to the capacitance value. COMSOL Multiphysics software was used for the simulation of the TENG performance.

The computer system can include a desktop computer, a workstation computer, a laptop computer, a netbook computer, a tablet, a handheld computer (including a smartphone), a server, a supercomputer, a wearable computer (including a SmartWatch™) or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C#, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft® .NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft® Corporation; the Mac OS X op-erating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of the medium. Combinations of media are also included within the scope of computer-readable media.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A self-powered system (100) for continuous real-time physiological signal monitoring, comprising:
a. a charging component (110) configured to generate power from movement of a user; and
b. one or more pressure sensors (120) operatively coupled to the charging component (110), configured to be applied to a user, measure one or more physiological signals of the user, and output one or more capacitance values, wherein the charging component (110) is further configured to power the one or more pressure sensors (120), wherein the one or more pressure sensors (120) comprise MXene.

2. The system (100) of claim 1, wherein the charging component (110) comprises an MXene-based triboelectric nanogenerator.

3. The system (100) of claim 1 further comprising a wearable component (130) configured to attach to a user.

4. The system (100) of claim 3, wherein the wearable component (130) is configured to attach to a wrist of the user.

5. The system (100) of claim 1 further comprising a capacitance-to-digital converter (CDC) (140) operatively coupled to the one or more pressure sensors (120) and the charging component (110), configured to convert the one or more capacitance values into one or more digital signals.

6. The system (100) of claim 5 further comprising a communication component (150) operatively coupled to the CDC (140) and an external source (170), configured to transmit the one or more digital signals to the external source (170).

7. The system (100) of claim 6, wherein the communication component (150) comprises a microcontroller unit (MCU) and a near field communication (NFC) chip further configured to be charged by an external charging source (180).

8. The system (100) of claim 7, wherein the external charging source (180) comprises a smart device or a wireless battery.

9. The system (100) of claim 5, wherein the external source (170) comprises a smart device, a personal computing device, a cloud server, or a physical server.

10. The system (100) of claim 1 further comprising one or more light-emitting diodes (LEDs) (160) operatively coupled to the one or more pressure sensors (120) and the charging component (110), configured to actuate in response to the movement of the user.

11. A self-powered system (100) for continuous real-time physiological signal monitoring, comprising:
a. a wearable component (130) configured to attach to a user;
b. a charging component (110) coupled to the wearable component (130), wherein the charging component (110) is configured to generate power from movement of the user;
c. one or more pressure sensors (120) coupled to the wearable component (130) and the charging component (110), configured to measure one or more physiological signals of the user and output one or more capacitance values, wherein the one or more pressure sensors (120) comprise MXene;
d. a capacitance-to-digital converter (CDC) (140) operatively coupled to the one or more pressure sensors (120) and the charging component (110), configured to convert the one or more capacitance values into one or more digital signals; and
e. a communication component (150) operatively coupled to the CDC (140) and an external source (170), configured to transmit the one or more digital signals to the external source (170).

12. The system (100) of claim 11, wherein the charging component (110) comprises an MXene-based triboelectric nanogenerator.

13. The system (100) of claim 11, wherein the wearable component (130) is configured to attach to a wrist of the user.

14. The system (100) of claim 13, wherein the communication component (150) comprises a microcontroller unit (MCU) and a near field communication (NFC) chip further configured to be charged by an external charging source (180).

15. The system (100) of claim 14, wherein the external charging source (180) comprises a smart device or a wireless battery.

16. The system (100) of claim 11, wherein the external source (170) comprises a smart device, a personal computing device, a cloud server, or a physical server.

17. The system (100) of claim 11 further comprising one or more light-emitting diodes (LEDs) (160) operatively coupled to the one or more pressure sensors (120) and the charging component (110), configured to actuate in response to the movement of the user.

18. A self-powered system (100) for continuous real-time physiological signal monitoring comprising:
a. a wearable component (130) configured to attach to a user;
b. a charging component (110) comprising a MXene-based triboelectric nanogenerator, coupled to the wearable component (130), wherein the charging component (110) is configured to generate power from movement of the user;
c. one or more pressure sensors (120) coupled to the wearable component (130) and the charging component (110), configured to measure one or more physiological signals of the user and output one or more capacitance values, wherein the one or more pressure sensors (120) comprise MXene;
d. a capacitance-to-digital converter (CDC) (140) operatively coupled to the one or more pressure sensors (120) and the charging component (110), configured to convert the one or more capacitance values into one or more digital signals;
e. a communication component (150) comprising a microcontroller unit (MCU) and a near field communication (NFC) chip, operatively coupled to the CDC (140) and an external source (170), configured to transmit the one or more digital signals to the external source (170) and further configured to be charged by an external charging source (180); and
f. one or more light-emitting diodes (LEDs) (160) operatively coupled to the one or more pressure sensors (120) and the charging component (110), configured to actuate in response to the movement of the user.
